(19) United States
(12) Patent Application Publication
(10) Pub. No.: US 2023/0398145 A1
(43) Pub. Date: Dec. 14, 2023

(54) METHOD OF INDUCING AN IMMUNE RESPONSE

(71) Applicant: Ravi PONNAPPAN, Moorestown, NJ (US)

(72) Inventor: Ravi PONNAPPAN, Moorestown, NJ (US)

(21) Appl. No.: 17/836,845

(22) Filed: Jun. 9, 2022

(51) Int. Cl. A61K 35/12; A61K 9/00; A61P 37/04

(52) U.S. Cl. CPC A61K 35/12; A61K 9/0019; A61P 37/04

(57) ABSTRACT

A method of inducing an immune response in a patient is provided. The method comprising biopsying a malignant tissue sample from a patient, freezing the malignant tissue sample to produce a frozen malignant tissue sample, mechanically pulverizing the frozen malignant tissue sample, and injecting the mechanically pulverized malignant tissue sample into said patient.

### BACKGROUND OF THE DISCLOSURE

Exemplary embodiments of the subject disclosure relate generally to the field of cancer treatment. Specifically, the subject disclosure relates to a method of inducing an immune response for treating cancer.

Cancer is the second leading cause of death in the United States right after heart disease. Even though there have been great advances in the areas of cancer research and treatments, much is still unknown in the field of cancer treatment.

More than 16.9 million Americans with a history of invasive cancer were alive in 2019, most of whom were diagnosed years ago and have no current evidence of the disease. More recently, the American Cancer Society has predicted that an estimated 1.9 million new cancer diagnoses are expected in 2021, with approximately 608,570 American lives taken as a result. Unfortunately, these staggering numbers only represent a small fraction of the total cancer diagnoses and deaths occurring throughout the world. As such, scientists, physicians, and clinical research teams have been meticulously researching different ways to combine current and future cancer treatment methods to increase survival rates and decrease metastasis.

As a result of this push towards more effective and efficient cancer treatments, scientists, physicians, and clinical research teams began to further explore the science behind immunotherapy. In this form of cancer treatment, the body's immune system is utilized to fight cancer. In other words, the patient's immune system is either activated or suppressed to combat the specific form of cancer they are fighting. For example, there is a form of immunotherapy called T cell therapy that utilizes cancer-specific T cells grown outside of the body and subsequently injected into the patient. Once injected into the patient, the cancer-specific T cells target the cancer, thereby inducing the body's natural immune response.

Unfortunately, issues do arise when developing these T cell immunotherapy programs as monetary, temporal, and scientific obstacles and restrictions are common. Beginning a T cell therapy program involves millions of dollars in investments and decades in research and development. In terms of the scientific deficiencies, growing viable T cells is a difficult task as temperature, pressure, humidity, and sterilization factors significantly alter the final results. This once again brings in the monetary and temporal factors as T cells have specific incubation periods and harvest times; meaning, failure to adhere to the specific incubation periods and harvest times will result in a nonviable T cell harvest and millions of dollars in waste.

Appreciating the deficiencies resulting from the monetary, temporal, and scientific obstacles of immunotherapy; scientists, physicians, and clinical research teams have begun another push to develop a low cost and more efficient way to induce an immune response in the treatment of cancer. In the present disclosure, the method of inducing an immune response remediates the monetary, temporal, and scientific impediments typically experienced by traditional immunotherapy methods. Overall, there is still a need for a cancer treatment method that is both cost-effective and efficient.

### BRIEF SUMMARY OF THE DISCLOSURE

In accordance with an exemplary embodiment of the subject disclosure, there is provided a method of inducing an immune response in a patient comprising biopsying a malignant tissue sample from a patient; freezing the malignant tissue sample to produce a frozen malignant tissue sample; mechanically pulverizing the frozen malignant tissue sample; and injecting the mechanically pulverized malignant tissue sample into said patient.

According to an aspect, the method of inducing an immune response further comprises the step of mixing the mechanically pulverized malignant tissue sample in a solvent.

According to an aspect of the method of inducing an immune response, the step of freezing the malignant tissue sample comprises freezing the malignant tissue sample with liquid nitrogen.

According to an aspect of the method of inducing an immune response, the step of mechanically pulverizing the frozen malignant tissue sample comprises crushing the frozen malignant tissue sample via a crusher tool.

According to another exemplary embodiment, the subject disclosure provides a method of inducing an immune response in a patient, the method comprising biopsying a tissue sample from a patient to produce a malignant tissue sample; freezing the malignant tissue sample; exposing epitopes of a mechanically pulverized malignant tissue sample while the tissue sample is frozen; and injecting the malignant tissue sample with exposed epitomes into said patient.

According to an aspect, the method of inducing an immune response further comprising the step of mixing the mechanically pulverized malignant tissue sample in a solvent.

According to an aspect of the method of inducing an immune response, the step of freezing the malignant tissue sample comprises freezing the malignant tissue sample with liquid nitrogen.

According to an aspect of the method of inducing an immune response, the step of exposing epitopes of the tissue sample comprises mechanical pulverization of the frozen malignant tissue sample.

According to another exemplary embodiment, the subject disclosure provides a kit for inducing an immune response, the kit comprising: a blade, a knife, or an extraction tool for retrieving a tissue sample; a sterile container for storing a tissue sample; a sterile crusher tool to mechanically pulverize a tissue sample; and a syringe for injecting a mechanically pulverized tissue sample into a patient.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIG. 1 is a flowchart of a method of inducing an immune response in a patient in accordance with an exemplary embodiment of the subject disclosure;

FIG. 2 is a flowchart of an exemplary process of freezing the biopsied malignant tissue sample with liquid nitrogen;

FIG. 3 is a flowchart of an exemplary method of mechanically pulverizing the frozen malignant tissue sample via a crusher tool;

FIGS. 4A-4C illustrate exemplary mechanical crushers applicable to the subject disclosure; and

FIG. 5 is an exemplary embodiment of a kit for inducing an immune response in a patient in accordance with another exemplary embodiment of the subject disclosure.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

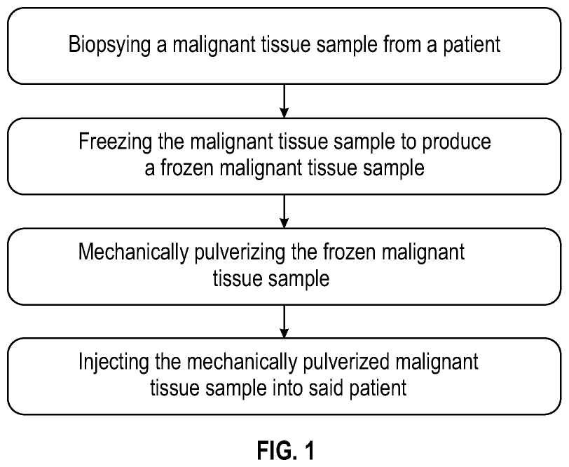

The present disclosure is directed to a method of inducing an immune response in a patient. The method includes biopsying a malignant tissue sample from a patient, freezing the malignant tissue sample to produce a frozen malignant tissue sample, mechanically pulverizing the frozen malignant tissue sample, and injecting the mechanically pulverized malignant tissue sample into the patient. An overview of the method for inducing an immune response is illustrated in FIG. 1.

Before biopsying the malignant tissue, the target tissue is first identified as benign or malignant. Once identified as being malignant, the malignant tissue is ready to be biopsied. The biopsy procedure can be carried out by a blade, a knife, or other biopsy extraction tool. The biopsy procedure itself can be conducted by typical biopsy methods, such as, a needle biopsy, a punch biopsy, a shave biopsy, a core needle biopsy, a vacuum-assisted biopsy, an excisional biopsy, an endoscopic biopsy, or laparoscopic biopsy or any other biopsy procedure sufficient to obtain a sufficient tissue sample.

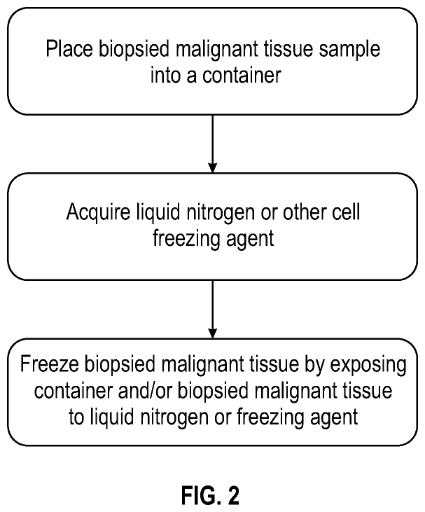

After the malignant tissue sample has been biopsied and retrieved, it can be transferred to a container or other suitable receptacle, e.g., sterile test tube, petri dish, or beaker. The container, including the sample, is then frozen to about −78.5 degrees C. to about −196 degrees C. e.g., by exposure to liquid nitrogen to freeze the tissue sample. This can be achieved by placing the entire container in liquid nitrogen or pouring liquid nitrogen over the container. Alternatively, the sample itself may be directly exposed to the liquid nitrogen. The container or sample is exposed to liquid nitrogen a sufficient amount of time to completely freeze the sample to about −80° C. Alternatively, instead of liquid nitrogen with a temperature of −196° C., the sample can be frozen by other means, such as dry ice with a temperature of −78.5° C., −80° C. ethanol, or by being placed in a −80° C. Freezer. An overview of the method for freezing the malignant tissue sample is illustrated in FIG. 2.

Alternatively, prior to freezing the malignant tissue sample, a solvent may be added to the malignant tissue sample. The addition of the solvent assists in protecting the malignant tissue sample from damage that could occur during the freezing process. The process of adding the solvent to the malignant tissue sample may utilize traditional cryoprotection solvents such as: dimethyl sulfoxide (DMSO), ethylene glycol, or glycerol and other solvents sufficient for such intended use.

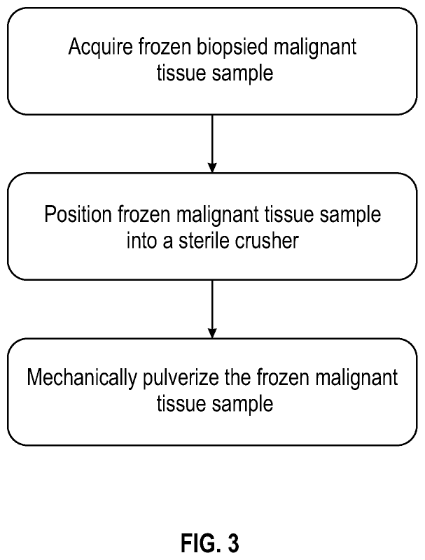

After freezing the malignant tissue sample, the sample is mechanically pulverized by a crusher, e.g., a sterile crusher. The mechanical pulverization utilizes mechanical means to pulverize the malignant tissue sample instead of traditional chemical means. The mechanical pulverization of the tissue sample produces a pulverized malignant tissue sample which results in the pulverized tissue sample exposing epitopes. That is, the frozen tissue sample is mechanically pulverized until epitopes are exposed. An overview of the method for mechanically pulverizing the malignant tissue sample is illustrated in FIG. 3.

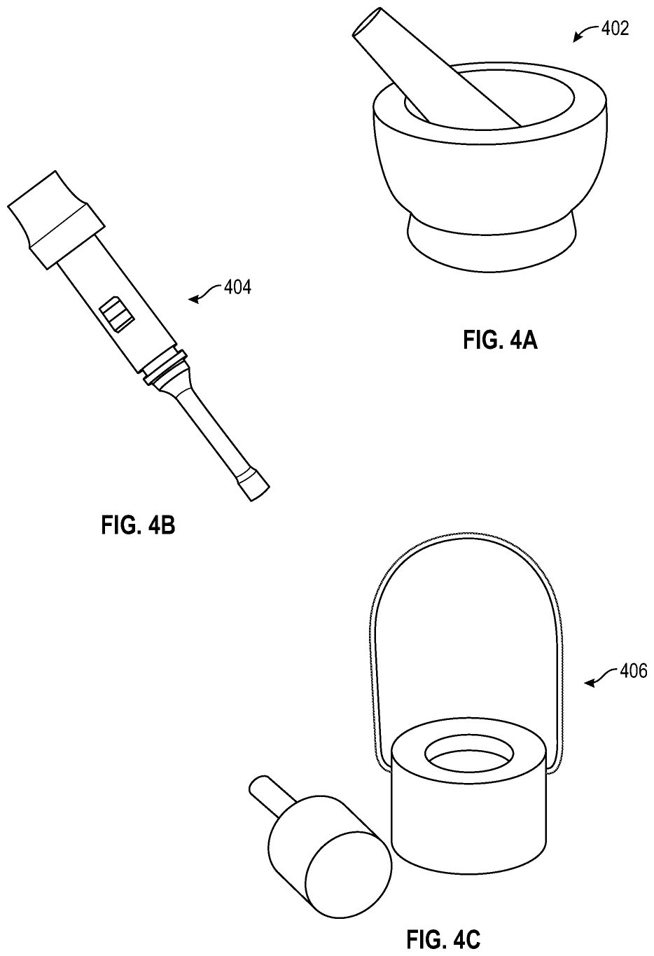

The sterile crusher may take the form of a mortar and pestle 402, a mechanical pulverizer 404, a mechanical crusher 406, a mechanical jaw crusher, and the like. Exemplary mechanical crushers applicable to the present disclosure are illustrated in FIGS. 4A-4C.

Following the mechanical pulverization of the frozen malignant tissue sample, the mechanically pulverized malignant tissue sample is thawed. After the mechanically pulverized malignant tissue sample is thawed, it can be tested for viability. Upon a nonviable test result, the thawed, nonviable, mechanically pulverized, malignant tissue sample is administered to the patient. This can be accomplished by placing the mechanically pulverized malignant tissue sample in a syringe 512 and injecting it into the patient. Additional methods for administering the tissue sample to a patient can include Intravenous (IV) injections and other techniques known in the art.

The volume of the injection is based upon a multitude of factors that will depend upon the patient and the type of cancer the treatment is attempting to combat. Therefore, the volume administered relies upon the direction of the treating physician. Exemplary volumes of the tissue sample to be administered e.g., via an injection, can range from 1-2 microliters, 1-500 microliters, 250-500 microliters, and 500-1,000 microliters. Exemplary volumes of the injection having the tissue sample for administration into a patient can range from 1-500 microliters, 250-500 microliters, 500-1,000 microliters, 1-1.5 ml, 1-2 ml, 1-3 ml, 1-4 ml, 1-5 ml and 5-10 ml.

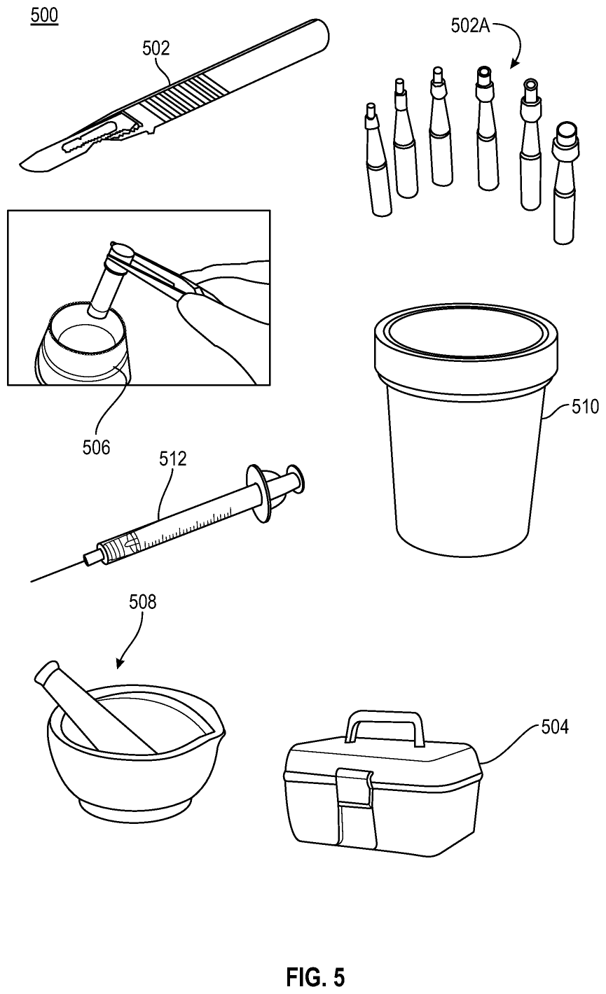

Referring to FIG. 5, in accordance with another exemplary embodiment of the subject disclosure there is provided a kit 500 for inducing an immune response in a patient. The kit includes a blade, a knife 502, an extraction tool 502A, or any other device capable of retrieving a tissue sample; a container 504 to store the tissue sample; a substance or mechanism 506 to freeze the tissue sample e.g., liquid nitrogen; a mechanical pulverizer 508 to mechanically pulverize the frozen tissue sample e.g., a mortar and pestle; a second sterile container 510 for thawing the tissue sample; and a syringe 512 for injecting the tissue sample into the patient.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims

I claim:

1. A method of inducing an immune response in a patient, the method comprising:
biopsying a malignant tissue sample from a patient;
freezing the malignant tissue sample to produce a frozen malignant tissue sample;
mechanically pulverizing the frozen malignant tissue sample; and
injecting the mechanically pulverized malignant tissue sample into said patient.

2. The method of inducing an immune response of claim 1, further comprising the step of mixing the mechanically pulverized malignant tissue sample in a solvent.

3. The method of inducing an immune response of claim 1, wherein the step of freezing the malignant tissue sample comprises freezing the malignant tissue sample with liquid nitrogen.

4. The method of inducing an immune response of claim 1, wherein the step of mechanically pulverizing the frozen malignant tissue sample comprises crushing the frozen malignant tissue sample via a crusher tool.

5. A method of inducing an immune response in a patient, the method comprising:
biopsying a tissue sample from a patient to produce a malignant tissue sample;
freezing the malignant tissue sample;
exposing epitopes of a mechanically pulverized malignant tissue sample while the tissue sample is frozen; and
injecting the malignant tissue sample with exposed epitomes into said patient.

6. The method of inducing an immune response of claim 5, further comprising the step of mixing the mechanically pulverized malignant tissue sample in a solvent.

7. The method of inducing an immune response of claim 5, wherein the step of freezing the malignant tissue sample comprises freezing the malignant tissue sample with liquid nitrogen.

8. The method of inducing an immune response of claim 5, wherein the step of exposing epitopes of the tissue sample comprises mechanical pulverization of the frozen malignant tissue sample.

9. A kit for inducing an immune response, the kit comprising:
a blade, a knife, or an extraction tool for retrieving a tissue sample;
a sterile container for storing a tissue sample;
a sterile crusher tool to mechanically pulverize a tissue sample; and
a syringe for injecting a mechanically pulverized tissue sample into a patient.
